(19) United States
(12) Patent Application Publication
(10) Pub. No.: US 2003/0086868 A1
(43) Pub. Date: May 8, 2003

(54) Actinium-225 complexes and conjugates for radioimmunotherapy

(76) Inventors: Dangshe Ma, Millwood, NY (US); Michael R. McDevitt, Bronx, NY (US); David A. Scheinberg, New York, NY (US); Jaime Simon, Angleton, TX (US); Garry E. Kiefer, Lake Jackson, TX (US); R. Keith Frank, Lake Jackson, TX (US); Gyongyi Gulyas, Lake Jackson, TX (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/031,792

(22) PCT Filed: Feb. 23, 2001
(86) PCT No.: PCT/US01/05927

(51) Int. Cl. A61K 51/00; C07F 5/00

(52) U.S. Cl. 424/1.65; 534/11; 540/474

(57) ABSTRACT

Actinium-225 (²²⁵Ac) complexes with functionalized chelants of the formula (I) wherein; each Q is independently hydrogen or ((CHR⁵)ₚCO₂R; Q¹ is hydrogen or (CHR⁵)_{w}CO₂R; each R independently is hydrogen, benzyl or C₁-C₄ alkyl; with the proviso that at least two of the sum of Q and Q¹ must be other than hydrogen, each R⁵ independently is hydrogen; C₁-C₄ alkyl or (C₁-C₂ alkyl)phenyl; X and Y are each independently hydrogen or may be taken with an adjacent X and Y to form an additional carbon—carbon bond; n is 0 or 1; m is an integer from 0 to 10 inclusive; p is 1 or 2; r is 0 or 1; w is 0 or 1; with the proviso that n is only 1 when X and/or Y form an additional carbon to carbon bond, and the sum of r and w is 0 or 1; L is a linker/spacer group covalently bonded to, and replaces one hydrogen atom of one of the carbon atoms to which it is joined, said linker/spacer group being represented by the formula (1) wherein s is an integer of 0 or 1; t is an integer of 0 to 20 inclusive; R¹ is an electrophilie or nucleophilic moiety which allows for covalent attachment to an antibody or fragment thereof, or synthetic linker which can be attached to an antibody or fragment thereof, or precursor thereof; and Cyc represents a cyclic aliphatic moiety, aromatic moiety, aliphatic heterocyclic moiety, or aromatic heterocyclic moiety, each of said moieties optionally substituted with one or more groups which do not interfere with binding to an antibody or antibody fragment; with the proviso that when s, t, m, r, and n are 0, then R¹ is other than carboxyl; their pharmaceutically acceptable salts, their conjugates and the use thereof for radioimmunotherapy is disclosed.

### FIELD OF THE INVENTION

This invention relates to actinium-225 (²²⁵Ac) complexes with fuctionalized chelants, their conjugates and their use for radioimmunotherapy.

### BACKGROUND OF THE INVENTION

The use of radionuclides complexed with suitable chelants, as well as their conjugates (that is, such complexes covalently attached to a biologically active carrier, for example, protein) for diagnosis of cancer and/or therapeutic treatment of cancer in mammals is known. These biochemically engineered molecules provide the tumor specificity and the radioisotope provides potent cytotoxicity. See, for example, U.S. Pat. Nos. 4,897,254; 5,342,925; 5,435,990; 5,652,361; 5,696,239; and 5,756,065.

It has been recognized that antibody-targeted alpha particles would allow extraordinary potent, single cell-specific killing with minimal toxicity to normal cells or the patient. The use of alpha particles as an alternative to more traditional classes of radiation is derived from the particle's kinetic characteristics and the radioactive half-life of their source isotope, as well as from the properties of the target-selective carrier moiety for the source isotope. The use of alpha emitting radionuclides is highly desirable for the following reasons: (a) a single atom can kill a cell making them hundreds to thousands of times more potent than even the most potent toxins or drugs; (b) the range of alpha particles is only about 50 microns, so that adjacent tissues are not harmed; (c) the chelated atoms on humanized antibodies are unlikely to be immunogenic and can be repeatedly dosed; (d) the radioactive atoms decay to harmless stable atoms; (e) killing can occur from inside or outside of the cell; (f) killing is by apoptosis and by double stranded DNA breaks and repair is not likely.

Specific cytotoxic effects of “alpha particle-emitting radioimmunoconjugates” have been demonstrated in several experimental systems. Specific in vitro cell-killing has been demonstrated against a human epidermoid cell line using ²¹³Bi- and ²²⁵Ac-containing immunoconjugates, see, for example, Kaspersen et al, Nuclear Medicine Communications, Vol. 15, pp. 468-476 (1995). Efficient and specific cell kill by the ²¹²Bi-labeled anti-Tac (CD25) monoclonal antibody has been demonstrated against an adult T-cell leukemia cell line in vitro, see, for example, R. W. Kozak et al, Proc. Natl. Acad. Sci. USA, Vol. 83, pp. 474-478 (1986). In other experiments, mice inoculated intraperitoneally with the murine tumor line EL-4 were cured of their ascites after intraperitoneal injection of 150 μCi of a ²¹²Bi-labeled antibody conjugate, see, for example, R. M. Macklis et al, Science, Vol. 240, pp. 1024-1026 (1988).

Potential for use of ²²⁵Ac in radiotherapy of cancer has also been recognized due to its favorable properties. This isotope decays with a radioactive half-life of 10 days into a cascade of short-lived alpha and beta-emitting isotopes. See, for example, M. W. Geerlings et al, Nuclear Medicine Communications, Vol. 14, pp. 121-125 (1993) and Kaspersen et al, Nuclear Medicine Communications, Vol. 15, pp. 468-476 (1995). However, the use of ²²⁵Ac in radioimmunotherapy has been hampered due to its toxicity and lack of a suitable carrier which will deliver it to the targeted cells.

In an effort to reduce the toxicity of ²²⁵Ac, numerous chelating agents such as, for example, 1,4,7,10-tetraazacyclododecane-1,4,7,10-tetraacetic acid (DOTA), diethylenetriaminepentaacetic acid (DTPA), ethylenediaminetetracetic acid (EDTA), 1,4,7,10,13-pentaazacyclo-pentadecane-1,4,7,10,13-pentaacetic acid (PEPA), and 1,4,7,10,13,16-hexaazacyclohexadecane-1,4,7,10,13,16-hexaacetic acid (HEHA) have been complexed with ²²⁵Ac and evaluated in vivo for toxicity and stability. However, the toxicity of these complexes has proved to be still substantial.

G. J. Beyer et al, Isotoperpraxis, Vol. 26, pp. 111-114 (1990), has evaluated the in vivo uptake of ²²⁵Accitrate and compared it to ¹⁶⁹Yb-citrate. This study has found that ²²⁵Ac-citrate had more efficient blood clearance, greater liver uptake, and lower bone uptake than ¹⁶⁹Yb-citrate.

G. J. Beyer et al, Nucl Med. & Biol., Vol. 24, pp. 367-372 (1997), has evaluated EDTMP (ethylenediaminetetra-methylenephosphonic acid) as a chelant for ²²⁵Ac. The study has found that EDTMP, depending on its concentration, reduces the liver uptake. However, the liver uptake of ²²⁵Ac-EDTMP is still substantial and excretion of ²²⁵Ac-EDTMP is poor. The study has also suggested that greater efficacy in endoradionuclide therapy of bone metastasis can be expected with the use of ²²⁵Ac-EDTMP due to the alpha-radiation.

K. A. Deal et al, J. Med. Chem., Vol 42, pp. 298-2992 (1999), has evaluated biodistribution of a number of ²²⁵Ac chelates. It has been observed that the structure of the chelant has dramatic effect on biodistribution of ²²⁵Ac. HEHA (1,4,7,10,13,16-hexaazacyclohexadecane-1,4,7,10,13,16-hexaacetic acid) was the largest macrocyclic chelant. ²²⁵Ac readily formed a complex with HEHA. Exceptional in vivo stability and reduced toxicity has been observed for ²²⁵Ac-HEHA. This has been attributed to the large size and macrocyclic effect of HEHA.

Although various chelating agents were suggested and evaluated as carriers for ²²⁵Ac, up to now ²²⁵Ac has not been successfully chelated to an antibody and no successful therapeutic use of ²²⁵Ac in animals or humans has been reported presumably due to its inherent toxicity and/or stability problems of its complexes.

It would be desirable to provide complexes comprising ²²⁵Ac and functionalized chelants which are kinetically and thermodynamically inert for use in therapeutic applications.

It would also be desirable to provide conjugates of such ²²⁵Ac complexes with a biological molecule. The biological molecule in these conjugates would provide the tumor specificity and the ²²⁵Ac isotope would provide potent cytotoxicity.

Another desirable property of these conjugates includes physiological compatibility which would permit the ²²⁵Ac complex, if separated from its targeting, conjugated biological molecule in vivo, to be soluble in physiological fluids and thus be rapidly eliminated from the body.

### SUMMARY OF THE INVENTION

The present invention is directed to ²²⁵Ac complexes and their conjugates with a biological molecule. The ²²⁵Ac compelexes and conjugates of the present invention are useful for the treatment of cancer in mammals, especially humans.

More specifically, the present invention is directed to ²²⁵Ac complexes comprising a functionalized chelant compound of the formula (I):

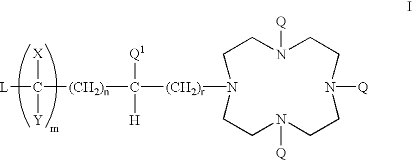

wherein:

each Q is independently hydrogen or (CHR⁵)ₚCO₂R;

Q¹ is hydrogen or (CHR⁵) C²R;

each R independently is hydrogen, benzyl or C₁-C₄ alkyl; with the proviso that at least two of the sum of Q and Q¹ must be other than hydrogen;

each R⁵ independently is hydrogen; C₁-C₄ alkyl or (C₁-C₂ alkyl)phenyl;

X and Y are each independently hydrogen or may be taken with an adjacent X and Y to form an additional carbon-carbon bond;

n is 0 or 1;

m is an integer from 0 to 10 inclusive;

p is 1 or 2;

r is 0 or 1;

w is 0 or 1;

with the proviso that n is only 1 when X and/or Y form an additional carbon-carbon bond, and the sum of r and w is 0 or 1;

L is a linker/spacer group covalently bonded to, and replaces one hydrogen atom of one of the carbon atoms to which it is joined, said linker/spacer group being represented by the formula

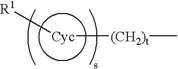

wherein

s is an integer of 0 or 1;

t is an integer of 0 to 20 inclusive;

R¹ is an electrophilic or nucleophilic moiety which allows for covalent attachment to an antibody or fragment of thereof, or synthetic linker which can be attached to an antibody or fragment thereof, or precursor thereof; and

Cyc represents a cyclic aliphatic moiety, aromatic moiety, aliphatic heterocyclic moiety, or aromatic heterocyclic moiety, each of said moieties optionally substituted with one or more groups which do not ineterfere with binding to a biologically active carrier;

with the proviso that when s, t, m, r, and n are 0, then R¹ is other than carboxyl;

or pharmaceutically acceptable salt thereof;

complexed with ²²⁵Ac.

The present invention is also directed to a conjugate comprising the aforementioned ²²⁵Ac complex covalently attached to a biological molecule.

The present invention also includes formulations having the conjugates of this invention and a pharmaceutically acceptable carrier, especially formulations where the pharmaceutically acceptable carrier is a liquid.

The present invention is also directed to a method of therapeutic treatment of a mammal having cancer which comprises administering to said mammal a therapeutically effective amount of the formulation of this invention.

Surprisingly, the ²²⁵Ac complexes and conjugates of this invention are relatively stable (that is, do not easily dissociate) and some display rapid clearance from the whole body and some non-target organs, such as liver and kidney. Additionally, the alpha-particles emitting ²²⁵Ac complexes and conjugates of this invention are expected to have several advantages over beta particle-emitting cytotoxic agents including higher energy and more potent emissions, less hazardous waste, expected lower effective dose, the potential for outpatient treatment, better retention at the target sites, and higher target to non-target radiation ratios.

### DETAILED DESCRIPTION OF THE INVENTION

As used herein, the term “²²⁵Ac complex” refers to a functionalized chelant compound of formula I complexed with ²²⁵Ac radionuclide.

As used herein, the term “²²⁵Ac conjugate” refers to ²²⁵Ac complex of the present invention that is covalently attached to a biological molecule.

As used herein, the term “mammal” means animals that nurish their young with milk secreted by mammary glands, preferably humans.

As used herein, the term “biological molecule” refers to any protein, antibody, antibody fragment, hormone, peptide, growth factor, antigen, hapten or any other carrier which functions in this invention to recognize a specific biological target site. Antibody and antibody fragment refers to any polyclonal, monoclonal, chimeric, human, mammalian, single chains, dimeric and tetrameric antibody or antibody fragment. Such biological molecule, when attached to a functionalized complex, serves to carry the attached ²²⁵Ac ion to specific targeted tissues. The term “antibody” refers to any polyclonal, monoclonal, chimeric antibody or heteroantibody. Preferably the antibodies used in the ²²⁵Ac conjugates of the present invention are monoclonal antibodies having high specificity for the desired cancer cells. Antibodies used in the present invention may be directed against, for example, cancer, tumors, leukemias, autoimune disorders involving cells of the immune system, normal cells that need to be ablated such as bone marrow and prostate tissue, virus infected cells including HIV, mycoplasma, differentiation and other cell membrane antigens, patogen surface antigens and any biologically active molecules. Some examples of antibodies are HuM195 (anti-CD33), CC-11, CC-46, CC-49, CC-49 F(ab′)₂, CC-83, CC-83 F(ab′)₂, and B72.3. Particularly preferred antibody for use in the practice of the present invention is HuM195. Antibody fragment includes Fab fragments and F(ab′)₂ fragments, and any portion of an antibody having specificity toward a desired epitope or epitopes. The antibodies which may be used in the ²²⁵Ac conjugates of the present invention can be prepared by techniques well known in the art. Highly specific monoclonal antibodies can be produced by hybridization techniques well known in the art, see, for example, Kohler and Milstein, Nature, 256, 495-497 (1975); and Eur. J. Immunol., 511-519 (1976).

As used herein, “pharmaceutically acceptable salt” means any salt of a compound of formula (I) which is sufficiently non-toxic to be useful in therapy of mammals. Representative of those salts, which are formed by standard reactions, from both organic and inorganic sources include, for example, sulfuric, hydrochloric, phosphoric, acetic, succinic, citric, lactic, maleic, fumaric, palmitic, cholic, palmoic, mucic, glutamic, d-camphoric, glutaric, glycolic, phthalic, tartaric, formic, lauric, steric, salicylic, methanesulfonic, bensenesulfonic, sorbic, picric, benzoic, cinnamic and other suitable acids. Also included are salts formed by standard reactions from both organic and inorganic sources such as ammonium, alkali metal ions, alkaline earth metal ions, and other similar ions. Preferred are the salts of the compounds of formula I where the salt is potassium, sodium, ammonium, or mixtures thereof.

As used herein, the term “therapeutically effective amount” means an amount of the ²²⁵Ac conjugate that produces a therapeutic effect on the disease treated. The therapeutically effective amount will vary depending on the mammal, the ²²⁵Ac conjugate and the method of its administration (for example, oral or parenteral). A person of ordinary skill in the art can determine the therapeutically effective amount of the ²²⁵Ac conjugate.

In the practice of the present invention the ²²⁵Ac conjugate may be administered per se or as a component of a pharmaceutically acceptable formulation.

Thus, the present invention may be practiced with the ²²⁵Ac conjugate being provided in pharmaceutical formulation, both for veterinary and for human medical use. Such pharmaceutical formulations comprise the active agent (the ²²⁵Ac conjugate) together with a physiologically acceptable carrier, excipient or vehicle therefore. The carrier(s) must be physiologically acceptable in the sense of being compatible with the other ingredient(s) in the formulation and not unsuitably deleterious to the recipient thereof. The ²²⁵Ac conjugate is provided in a therapeutically effective amount, as described above, and in a quantity appropriate to achieve the desired dose.

The formulations include those suitable for parenteral (including subcutaneous, intramuscular, intraperitoneal, and intravenous), oral, rectal, topical, nasal, or ophthalmic administration. Formulations may be prepared by any methods well known in the art of pharmacy. Such methods include the step of bringing the ²²⁵Ac conjugate into association with a carrier, excipient or vehicle therefore. In general, the formulation may be prepared by uniformly and intimately bringing the ²²⁵Ac conjugate into association with a liquid carrier, a finely divided solid carrier, or both, and then, if necessary, shaping the product into desired formulation. In addition, the formulations of this invention may further include one or more accessory ingredient(s) selected from diluents, buffers, binders, disintegrants, surface active agents, thickeners, lubricants, preservatives, and the like. In addition, a treatment regime might include pretreatment with non-radioactive carrier.

Injectable formulations of the present invention may be either in suspensions or solution form. In the preparation of suitable formulations it will be recognized that, in general, the water solubility of the salt is greater than the acid form. In soliution form the complex (or when desired the separate components) is dissolved in a physiologically acceptable carrier. Such carriers comprise a suitable solvent, preservatives such as free radical quenching agents, for example, ascorbic acid, benzyl alcohol or any other suitable molecule, if needed, and buffers. Useful solvents include, for example, water, aqueous alcohols, glycols, and phosphonate or carbonate esters. Such aqueous solutions contain no more than 50 percent of the organic solvent by volume.

Injectable suspensions are compositions of the present invention that require a liquid suspending medium, with or without adjuvants, as a carier. The suspending medium can be, for example, aqueous polyvinylpyrrolidone, inert oils such as vegetable oils or highly refined mineral oils, polyols, or aqueous carboxymethylcellulose. Suitable physiologically acceptable adjuvants, if necessary to keep the complex in suspension, may be chosen from among thickeners such as carboxymethylcellulose, polyvinylpyrrolidone, gelatin, and the alginates. Many surfactants are also useful as suspending agents, for example, lecithin, alkylphenol, polyethyleneoxide adducts, naphthalenesulfonates, alkylbenzenesulfonates, and polyoxyethylene sorbitane esters.

In the context of the present invention the terms “functionalized chelant” and “bifunctional chelant” are used interchangeably and refer to compounds which have the dual functionality of sequestering metal ions plus the ability to covalently bind a biological molecule having specificity for tumor cell epitopes or antigens. Such compounds are of great utility for therapeutic and diagnostic applications when they are, for example, complexed with radioactive metal ions and covalently attached to a specific antibody. These types of complexes have been used to carry radioactive metals to tumor cells which are targeted by the specificity of the attached antibody [see, for example, Mears et al., Anal. Biochem. 142, 68-74 (1984); Krejcarek et al., Biochem. And Biophys. Res. Comm. 77, 581-585 (1977)].

The functionalized chelant compounds of formula (I) useful in the practice of the present invention are known in the art. See, for example, U.S. Pat. Nos. 5,435,990 and 5,652,361.

Compounds of formula I where: R is hydrogen or methyl; n is 0; m is 0 through 5; r is 0; and L is a moiety of formula A:

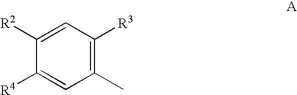

wherein:

R² is selected from the group consisting of hydrogen, nitro, amino, isothiocyanato, semicarbazido, thiosemicarbazido, carboxyl, bromoacetamido and maleimido;

R³ is selected from the group consisting of C₁-C₄ alkoxy, —OCH₂CO₂H, hydroxy and hydrogen; and

R⁴ is selected from the group consisting of hydrogen, nitro, amino, isothiocyanato, semicarbazido, thiosemicarbazido, carboxyl, bromoacetamido and maleimido;

with the proviso that R² and R⁴ cannot both be hydrogen but one of R² and R⁴ must be hydrogen; or a pharmaceutically acceptable salt thereof; are preferred functionalized chelants.

Preferred functionalized chelant compounds of formula I include also those compounds where Q¹ is hydrogen and L is represented by formula A as shown by formula II:

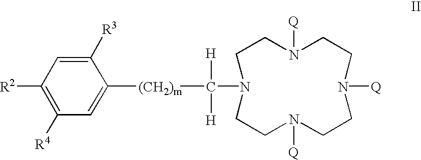

wherein:

each Q independently is hydrogen or CHR⁵COOR; with the proviso that at least two of Q must be other than hydrogen

each R independently is hydrogen benzyl or C₁-C₄ alkyl;

m is integer from 0 to 5 inclusive;

R² is selected from the group consisting of hydrogen, nitro, amino, isothiocyanato, semicarbazido, thiosemicarbazido, carboxyl, bromoacetamido and maleimido;

R³ is selected from the group consisting of C₁-C₄ alkoxy, —OCH₂COOH, hydroxy and hydrogen;

R⁴ is selected from the group consisting of hydrogen, nitro, amino, isothiocyanato, semicarbazido, thiosemicarbazido, carboxyl, bromoacetamido and maleimido;

each R⁵ independently is hydrogen or C₁-C₄ alkyl;

with the proviso that R² and R⁴ cannot both be hydrogen but one of R² and R⁴ must be hydrogen; or

a pharmaceutically acceptable salt thereof.

Additional preferred functionalized chelant compounds of formula I include those compounds where at least one Q is hydrogen and are represented by formula III:

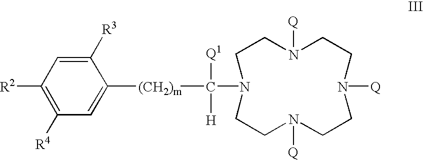

wherein:

each Q independently is hydrogen or CHR⁵COOR;

Q¹ is hydrogen or (CHR⁵)_{w}CO₂R; with the proviso that at least two the sum of Q and Q¹ must be other than hydrogen and one Q is hydrogen;

each R independently is hydrogen benzyl or C₁-C₄ alkyl;

m is integer from 0 to 5 inclusive;

w is 0 or 1;

R² is selected from the group consisting of hydrogen, nitro, amino, isothiocyanato, semicarbazido, thiosemicarbazido, carboxyl, bromoacetamido and maleimido;

R³ is selected from the group consisting of C₁-C₄ alkoxy, —OCH₂COOH, hydroxy and hydrogen;

R⁴ is selected from the group consisting of hydrogen, nitro, amino, isothiocyanato, semicarbazido, thiosemicarbazido, carboxyl, bromoacetamido and maleimido;

each R⁵ independently is hydrogen or C₁-C₄ alkyl;

with the proviso that R² and R⁴ cannot both be hydrogen but one of R² and R⁴ must be hydrogen; or

a pharmaceutically acceptable salt thereof.

Other preferred functionalized chelant compounds of formula I include compounds where Q¹ is CO₂R (w=0) and are represented by formula IV:

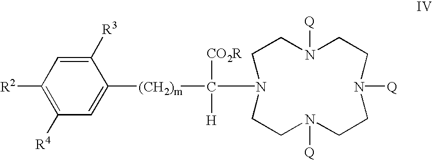

wherein:

each Q independently is hydrogen or CHR⁵ COOR; with the proviso that at least one Q must be other than hydrogen;

each R independently is hydrogen benzyl or C₁-C₄ alkyl;

m is integer from 0 to 5 inclusive;

R² is selected from the group consisting of hydrogen, nitro, amino, isothiocyanato, semicarbazido, thiosemicarbazido, carboxyl, bromoacetamido and maleimido;

R³ is selected from the group consisting of C₁-C₄ alkoxy, —OCH₂COOH, hydroxy and hydrogen;

R⁴ is selected from the group consisting of hydrogen, nitro, amino, isothiocyanato, semicarbazido, thiosemicarbazido, carboxyl, bromoacetamido and maleimido;

each R⁵ independently is hydrogen or C₁-C₄ alkyl;

with the proviso that R² and R⁴ cannot both be hydrogen but one of R² and R⁴ must be hydrogen; or

a pharmaceutically acceptable salt thereof.

The functionalized chelants of formula I useful in the practice of the present invention can be prepared by known methods. General synthetic approach to a twelve-membered macrocyclic, bifunctional chelant of the present invention as represented by formula I involves monofuctionalization of a free-base macrocycle (for example, 1,4,7,10-tetraazacyclododecane) at only one of the nitrogen atoms with an appropriate electrophile (for example, any appropriately substituted alpha-halocarboxylic acid ester). This electrophile must possess a suitable linker moiety which would allow covalent attachment of bifunctional ligand to a biological molecule. Various synthetic routes to functionalized chelants of formula I have been described U.S. Pat. Nos. 5,435,990 and 5,652,361, both incorporated herein by reference.

The method of obtaining ²²⁵Ac radionuclide is not critical to the present invention. For example, ²²⁵Ac can be prepared in a cyclotron. ²²⁵Ac can be obtained in pure form from Department of Energy (DOE), U.S.A., and Institute for Transuranium Elements (ITU), Karlsruhe, Germany.

The ²²⁵Ac conjugates of the present invention can be prepared by first forming the complex and then binding the biological molecule. Thus, the process involves preparing or obtaining the ligand, forming the complex with ²²⁵Ac and then adding the biological molecule. Alternatively, the process may involve first conjugation of the ligand to the biological molecule and then the formation of the complex with ²²⁵Ac. Any suitable process that results in the formation of the ²²⁵Ac conjugates of this invention is within the scope of the present invention.

In the following examples, the following terms and conditions were used unless otherwise specified.

### GLOSSARY OF TERMS

Ab=antibody;

BFC=bifunctional chelant;

DOTA=1,4,7,10 tetraazacyclododecane-1,4,7,10-tetraacetic acid;

MeO-DOTA-NCS=1-[(2-methoxy-5-isothiocyanato-phenyl)-carboxymethyl]-4,7,10-triscarboxymethyl-1,4,7,10-tetraazacyclododecane;

TMAA=tetramethyl ammonium acetate buffer;

Sephadex C-25 resin is a cation exchange resin, sold by Pharmacia Inc.;

EDTA=ethylenediaminetetraacetic acid;

DTPA=diethylenetriaminepentaacetic acid;

TETA=1,4,8,11-tetraazacyclotetradecane-1,4,8,11-tetraacetic acid;

DOTPA=1,4,7,10-tetraazacyclododecane-1,4,7,10-tetrapropionic acid;

TETPA=1,4,8,11-tetraazacyclotetradecane-1,4,8,11-tetrapropionic acid;

DOTMP=1,4,6,10-tetraazacyclodecane-1,4,7,10-tetramethylenephosphonic acid.

General Experimental

Method of preparation of ²²⁵Ac conjugates: The preparation of ²²⁵Ac conjugates involved two steps. First, the ²²⁵Ac complex was prepared by mixing a solution of the functionalized chelant compound of formula I with the solution of ²²⁵Ac at pH of about 5-6 in a suitable buffer. The complex formation was tested using cation exchange chromatography. Then, the conjugation of the ²²⁵Ac complex to a biological molecule (suitably an antibody) was carried out at the pH of about 8.5 in the presence of a suitable buffer. The antibody and the antibody ²²⁵Ac complex conjugates were then separated from the unconjugated low molecular weight materials using gel filtration chromatography. The fraction of radioactivity associated with the antibody was then determined.

The γ emission counting was performed using a 3-inch×3-inch NaI well crystal utilizing the γ emission of ²²⁵Ac decay product 221Fr (half-life of 4.8 min.) at 218 KeV. Counting was carried out half an hour after sample preparation.

Method for determining yield and stability of ²²⁵Ac complexes and conjugates thereof: Instant Thin Layer Chromatography (ITLC) was utilized with either a 10 mM EDTA or 10 mM NaOH/9% NaCl solvent systems using ITLC SG strips (sold by Gelman Sciences company) to assess the complexation and conjugation efficiency of the DOTA-based bifunctional ²²⁵Ac conjugate with HuM195 antibody.

The following examples are provided to further illustrate the present invention, and should not be construed as limiting thereof.

### EXAMPLE 1

### Preparation of ²²⁵Ac-MeO-DOTA-NCS Complex

An aqueous solution of MeO-DOTA-NCS (35 p1; 0.31 mg/ml) was mixed with the ²²⁵Ac chloride solution (35 p1; 1.65 μCi/μl,) in 0.1M HCl. The pH was adjusted to about 5 using the TMAA buffer (130 μl, 0.2 M, pH about 6). Reaction mixture was incubated at about 50° C. for one hour. Complex formation was checked by cation exchange chromatography employing the Sephadex C-25 resin and it was determined that 99 percent of ²²⁵Ac was complexed.

### EXAMPLE 2

### Preparation of ²²⁵Ac-HuM195 Conjugate

HuM195 antibody solution (20 μl, 5 mg/ml) was added to the ²²⁵Ac complex solution (200 μl) prepared as described in Example 1. The pH was adjusted to about 8.5 using a NaHCO₃ buffer (85 μl, 0.1 M, pH=8.7). The molar ratios of the reactants used were as follows: MeO-DOTA-NCS/²²⁵Ac=6549; MeO-DOTA-NCS/HuM195=24; and HuM195 /²²⁵Ac=275. After 30 minutes incubation at 20° C. the protein and the small molecular weight components of the solution were separated by gel filtration chromatography using the Econo-Pack 10 DG gel filtration column. The extent of coupling was determined by γ emission counting. It was determined that 7.3 percent of the ²²⁵Ac complex was coupled to HuM195 antibody.

### EXAMPLE 3

### Conjugation of Antibodies HuM195 (anti-CD33) and B4 (anti-CD19) to MeO-DOTA-NCS

20 mg of HuM195 (or B4) monoclonal antibody solution was mixed with 0.05 M HEPES containing EDTA at about pH 8 and dialysed against 0.05 M HEPES buffer for 24 hours in an Amicon stirred cell dialysis unit to remove any metal ions associated with antibodies. A MeO-DOTA-NCS solution containing 3.38 mg of MeO-DOTA-NCS was added and allowed to conjugate with the antibody at room temperature for 24 hours. Then the reaction mixture was dialysed against a NaAc/NaCl buffer solution at about pH 7.0 for 24 hours to remove any unreacted MeO-DOTA-NCS. The immunoconjugate was recovered from the stirred cell and characterized using a size exclusion high pressure liquid chromatography (HPLC). This was compared to the native HuM195 or B4. Antibody concentration was determined by UV-absorption at 280 nM.

The immunoconjugates could be labeled readily with ¹¹¹In showing success of the conjugation reaction. For example, approximately 400 μCi of ¹¹¹In in 200 μl of 0.2 M HCl was mixed with 29 μl of ammonium acetate (3M) and 9 μl of l-ascorbic acid (150 mg/ml) to adjust the pH to 5.0 and then 0.5 mg of HuM195-MeO-DOTA-NCS immunoconjugate was added. The reaction was allowed to progress at 37° C. for 60 minutes. An 87% reaction yield was obtained.

### EXAMPLE 4

### Labeling of HuM195-MeO-DOTA Immunoconjugate with ²²⁵Ac

200 μCi of ²²⁵Ac in 0.2 M HCl was mixed with 700 μl metal free water and then buffered with 93 μl of NH₄Ac at about pH 6.5. Each of four 200 μl aliquots was mixed with 0 mg, 0.1 mg, 0.5 mg, 1 mg of HuM195-MeO-DOTA (prepared as in Example 3), respectively. The reaction tubes were placed into a 37° C. water bath. The ²²⁵Ac incorporation was monitored by TLC developed in 10 mM EDTA solvent at 3 hours. The ²²⁵Ac incorporation (percentage of activity remaining at origin) data are given in Table 1 below for the various antibody concentrations and Ab:Ac ratios.

TABLE 1
²²⁵Ac Incorporation at 3 hr for three different
concentrations of the antibody

| | | | |
|---|---|---|---|
| Antibody (μM) | 1.6 | 8.1 | 16.1 |
| Ab to Ac Ratios | 174 | 870 | 1740 |
| % Incorporation | 7.1 | 23.1 | 51.8 |

### EXAMPLE 5

### Stability of ²²⁵Ac-HuM195-MeO-DOTA

The three ²²⁵Ac-HuM195-MeO-DOTA solutions from Example 4 were combined and challenged with 20 μl of 10 mM DTPA to remove unbounded metals. 100 μl of 1-ascorbic acid (150 mg/ml) was added as a radioprotection agent. The solution was purified through a 10-DG desalting column (Bio-Rad company) to separate ²²⁵Ac-HuM195-MeO-DOTA from unreacted ²²⁵Ac (in DTPA form). The purified ²²⁵Ac-HuM195-MeO-DOTA was subjected to a stability study in human serum.

The purified ²²⁵Ac-HuM195-MeO-DOTA was assessed for stability in different media such as 1% and 25% albumin (human) and 1% and 25% human serum at 37° C. Overall stability half-lives of ²²⁵Ac-HuM195-MeO-DOTA in either albumin (human) at 4° C. or human serum at 37° C. exceeded 150 days.

### EXAMPLE 6

### ²²⁵Ac-HuM195-MeO-DOTA in-vitro Cell Kill

A cell-based immunoreactivity study (binding of labeled antibody to antigen excess) has shown that ²²⁵Ac labeled HuM195 antibody (²²⁵Ac-HuM195-MeO-DOTA) is still immunoreactive (˜70%). The potency and specificity of ²²⁵Ac-HuM195-MeO-DOTA was then evaluated in-vitro as a function of specific activity and activity concentration on antigen positive and negative cell lines. The LD₅₀ in a 5-day assay was ˜0.3 nCi/ml at a specific activity of 0.035 Ci/g which is 3 log more potent than the similar ²¹³Bi alpha emitting agent with much high specific activity (see Nikula et al, J Nucl Med 1999; 40, 166-176). These data (derived from 3H-thymidine incorporation) are plotted below in FIG. 3. The LD₅₀ in a 2-day assay is about 1.4 nCi/ml for positive cell line and about 28 nCi/ml for negative cell line which demonstrates the specificity. Internalization of isotope into target cells was also demonstrated and more than 50% was internalized in the target cells in 5 hours which is crucial to control the fate of daughter isotopes. This preliminary study suggests that ²²⁵Ac-HuM195-MeO-DOTA conjugates are useful clinically as a way to target alpha particles to kill cells.

[image]

### EXAMPLE 7

### In vivo Biodistribution

The in vivo biodistribution of free ²²⁵Ac acetate, ²²⁵Ac-DOTA and ²²⁵Ac-HuM195-MeO-DOTA was studied in nu/nu mice by intraperitoneal injection of approximately 2 μCi of ²²⁵Ac of each compound in 400 μl. It was demonstrated that a different pattern of distribution existed (see Table 2 below) for the three agents. The ²²⁵Ac-DOTA was excreted very quickly and most activity was cleared in less than 40 minutes. ²²⁵Ac in acetate was held up in the liver and bone but cleared from blood. The ²²⁵Ac-HuM195-MeO-DOTA has longer blood circulation time and less bone uptake over the period of 5 days. These data indicated ²²⁵Ac-HuM195-MeO-DOTA is stable in vivo.

TABLE 2
Summary of the ²²⁵Ac Biodistribution (% dose/g) in nu/nu Mice

| | | | | | | | ²²⁵Ac-HuM195- | | |
|---|---|---|---|---|---|---|---|---|---|
| | | | | ²²⁵Ac-DOTA | | | DOTAHuM195- | | |
| | ²²⁵Ac Acetate | | | 40 | | | MeO-DOTA | | |
| Average | 1 d | 2 d | 5 d | min | 2 h | 18 h | 1 d | 2 d | 5 d |
| Blood | 0.1 | 0.2 | 0.0 | 3.0 | 1.8 | 1.1 | 10.1 | 10.2 | 4.5 |
| Kidneys | 3.8 | 3.2 | 2.8 | 4.9 | 3.5 | 3.1 | 4.6 | 5.2 | 4.6 |
| Liver | 46.6 | 43.1 | 68.5 | 4.5 | 4.6 | 5.8 | 8.8 | 10.0 | 20.7 |
| Bone | 13.3 | 12.0 | 17.1 | 3.9 | 4.2 | 4.9 | 3.8 | 4.1 | 5.3 |

What is claimed is:

1. An ²²⁵Ac complex comprising a functionalized chelant compound of the formula I 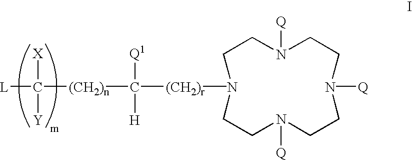
wherein:
each Q is independently hydrogen or (CHR⁵) CO₂R;
Q¹ is hydrogen or (CHR⁵)_{w}CO₂R;
each R independently is hydrogen, benzyl or C₁-C₄ alkyl; with the proviso that at least two of the sum of Q and Q¹must be other than hydrogen;
each R⁵ independently is hydrogen; C₁-C₄ alkyl or (C₁-C₂ alkyl)phenyl;
X and Y are each independently hydrogen or may be taken with an adjacent X and Y to form an additional carbon-carbon bond;
n is 0 or 1;
m is an integer from 0 to 10 inclusive;
p is 1 or 2;
r is 0 or 1;
w is 0 or 1;
with the proviso that n is only 1 when X and/or Y form an additional carbon to carbon bond, and the sum of r and w is 0 or 1;
L is a linker/spacer group covalently bonded to, and replaces one hydrogen atom of one of the carbon atoms to which it is joined, said linker/spacer group being represented by the formula 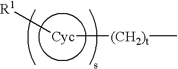
wherein
s is an integer of 0 or 1;
t is an integer of 0 to 20 inclusive;
R¹ is an electrophilic or nucleophilic moiety which allows for covalent attachment to an antibody or fragment of thereof, or synthetic linker which can be attached to an antibody or fragment thereof, or precursor thereof; and
Cyc represents a cyclic aliphatic moiety, aromatic moiety, aliphatic heterocyclic moiety, or aromatic heterocyclic moiety, each of said moieties optionally substituted with one or more groups which do not interfere with binding to an antibody or antibody fragment;
with the proviso that when s, t, m, r, and n are 0, then R¹ is other than carboxyl; or
a pharmaceutically acceptable salt thereof; complexed with ²²⁵Ac.

2. The ²²⁵Ac complex of claim 1 wherein the functionalized chelant is a compound of formula II 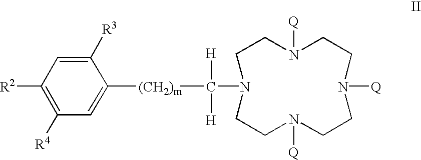
wherein:
each Q independently is hydrogen or CHR⁵COOR; with the proviso that at least two of Q must be other than hydrogen
each R independently is hydrogen benzyl or C₁-C₄ alkyl;
m is integer from 0 to 5 inclusive;
R² is selected from the group consisting of hydrogen, nitro, amino, isothiocyanato, semicarbazido, thiosemicarbazido, carboxyl, bromoacetamido and maleimido;
R³ is selected from the group consisting of C₁-C₄ alkoxy, —OCH₂COON, hydroxy and hydrogen;
R⁴ is selected from the group consisting of hydrogen, nitro, amino, isothiocyanato, semicarbazido, thiosemicarbazido, carboxyl, bromoacetamido and maleimido;
each R⁵ independently is hydrogen or C₁-C₄ alkyl;
with the proviso that R² and R⁴ cannot both be hydrogen but one of R² and R⁴ must be hydrogen; or a pharmaceutically acceptable salt thereof.

3. The ²²⁵Ac complex of claim 1 wherein the functionalized chelant is a compound of formula III 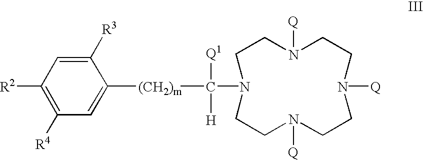
wherein:
each Q independently is hydrogen or CHR⁵COOR;
Q¹ is hydrogen or (CHR⁵)_{w}CO₂R; with the proviso that at least two the sum of Q and Q¹ must be other than hydrogen and one Q is hydrogen;
each R independently is hydrogen benzyl or C₁-C₄ alkyl;
m is integer from 0 to 5 inclusive;
w is 0 or 1;
R² is selected from the group consisting of hydrogen, nitro, amino, isothiocyanato, semicarbazido, thiosemicarbazido, carboxyl, bromoacetamido and maleimido;
R³ is selected from the group consisting of C₁-C₄ alkoxy, —OCH₂COOH, hydroxy and hydrogen;
R⁴ is selected from the group consisting of hydrogen, nitro, amino, isothiocyanato, semicarbazido, thiosemicarbazido, carboxyl, bromoacetamido and maleimido;
each R⁵ independently is hydrogen or C₁-C₄ alkyl; with the proviso that R² and R⁴ cannot both be hydrogen but one of R and R⁴ must be hydrogen; or
a pharmaceutically acceptable salt thereof.

4. The ²²⁵Ac complex of claim 1 wherein the fuctionalized chelant is a compound of formula IV 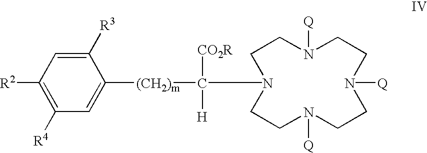
wherein:
each Q independently is hydrogen or CHR⁵COOR; with the proviso that at least one Q must be other than hydrogen;
each R independently is hydrogen benzyl or C₁-C₄ alkyl;
m is integer from 0 to 5 inclusive;
R² is selected from the group consisting of hydrogen, nitro, amino, isothiocyanato, semicarbazido, thiosemicarbazido, carboxyl, bromoacetamido and maleimido;
R³ is selected from the group consisting of C₁-C₄ alkoxy, —OCH₂COOH, hydroxy and hydrogen;
R⁴ is selected from the group consisting of hydrogen, nitro, amino, isothiocyanato, semicarbazido, thiosemicarbazido, carboxyl, bromoacetamido and maleimido;
each R⁵ independently is hydrogen or C₁-C₄ alkyl;
with the proviso that R² and R⁴ cannot both be hydrogen but one of R and R must be hydrogen; or a pharmaceutically acceptable salt thereof.

5. The ²²⁵Ac complex of claim 1 wherein the functionalized chelant compound is 1-[(2-methoxy-5-isothiocyanatophenyl)-carboxymethyl]-4,7,10-triscarboxymethyl-1,4,7,10-tetraazacyclododecane (MeO-DOTA-NCS).

6. An ²²⁵Ac conjugate comprising a functionalized chelant compound of the formula I 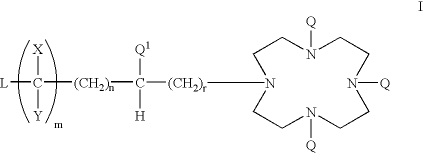
wherein:
each Q is independently hydrogen or (CHR⁵)ₚCO₂R;
Q¹ is hydrogen or (CHR⁵)_{w}CO₂R;
each R independently is hydrogen, benzyl or C₁-C₄ alkyl; with the proviso that at least two of the sum of Q and Q¹ must be other than hydrogen;
each R⁵ independently is hydrogen; C₁-C₄ alkyl or (C₁-C₂ alkyl)phenyl;
X and Y are each independently hydrogen or may be taken with an adjacent X and Y to form an additional carbon-carbon bond;
n is 0 or 1;
m is an integer from 0 to 10 inclusive;
p is 1 or 2;
r is 0 or 1;
w is 0 or 1;
with the proviso that n is only 1 when X and/or Y form an additional carbon-carbon bond, and the sum of r and w is 0 or 1;
L is a linker/spacer group covalently bonded to, and replaces one hydrogen atom of one of the carbon atoms to which it is joined, said linker/spacer group being represented by the formula 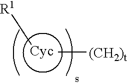
wherein
s is an integer of 0 or 1;
t is an integer of 0 to 20 inclusive;
R¹ is an electrophilic or nucleophilic moiety which allows for covalent attachment to an antibody or fragment of thereof, or synthetic linker which can be attached to an antibody or fragment thereof, or precursor thereof; and
Cyc represents a cyclic aliphatic moiety, aromatic moiety, aliphatic heterocyclic moiety, or aromatic heterocyclic moiety, each of said moieties optionally substituted with one or more groups which do not interfere with binding to an antibody or antibody fragment;
with the proviso that when s, t, m, r, and n are 0, then R¹ is other than carboxyl; or
pharmaceutically acceptable salt thereof;
complexed with ²²⁵Ac; and covalently attached to a biological molecule.

7. The ²²⁵Ac conjugate of claim 6 wherein the functionalized chelant is a compound of formula II 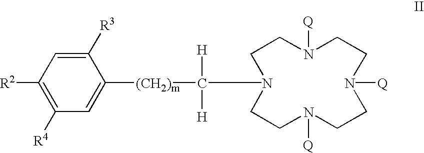
wherein:
each Q independently is hydrogen or CHR⁵COOR; with the proviso that at least two of Q must be other than hydrogen
each R independently is hydrogen benzyl or C₁-C₄ alkyl;
m is integer from 0 to 5 inclusive;
R² is selected from the group consisting of hydrogen, nitro, amino, isothiocyanato, semicarbazido, thiosemicarbazido, carboxyl, bromoacetamido and maleimido;
R³ is selected from the group consisting of C₁-C₄ alkoxy, -OCH₂COOH, hydroxy and hydrogen;
R⁴ is selected from the group consisting of hydrogen, nitro, amino, isothiocyanato, semicarbazido, thiosemicarbazido, carboxyl, bromoacetamido and maleimido;
each R⁵ independently is hydrogen or C₁-C₄ alkyl; with the proviso that R² and R⁴ cannot both be hydrogen but one of R² and R⁴ must be hydrogen; or
a pharmaceutically acceptable salt thereof.

8. The ²²⁵Ac conjugate of claim 6 wherein the functionalized chelant is a compound of formula III 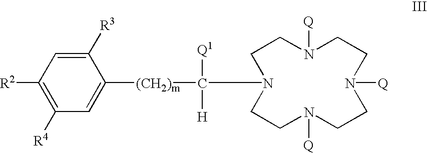
wherein:
each Q independently is hydrogen or CHR⁵COOR;
Q¹ is hydrogen or (CHR⁵)_{w}CO₂R; with the proviso that at least two the sum of Q and Q¹ must be other than hydrogen and one Q is hydrogen; each R independently is hydrogen benzyl or C₁-C₄ alkyl;
m is integer from 0 to 5 inclusive;
w is 0 or 1;
R² is selected from the group consisting of hydrogen, nitro, amino, isothiocyanato, semicarbazido, thiosemicarbazido, carboxyl, bromoacetamido and maleimido;
R³ is selected from the group consisting of C₁-C₄ alkoxy, -OCH₂COOH, hydroxy and hydrogen;
R⁴ is selected from the group consisting of hydrogen, nitro, amino, isothiocyanato, semicarbazido, thiosemicarbazido, carboxyl, bromoacetamido and maleimido;
each R⁵ independently is hydrogen or C₁-C₄ alkyl;
with the proviso that R² and R⁴ cannot both be hydrogen but one of R² and R⁴ must be hydrogen; or a pharmaceutically acceptable salt thereof.

9. The ²²⁵Ac conjugate of claim 6 wherein the functionalized chelant is a compound of formula IV 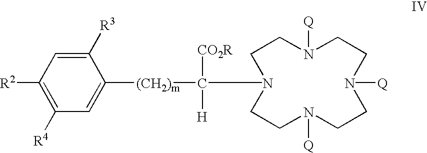
wherein:
each Q independently is hydrogen or CHR⁵COOR; with the proviso that at least one Q must be other than hydrogen;
each R independently is hydrogen, benzyl or C₁-C₄ alkyl;
m is integer from 0 to 5 inclusive;
R² is selected from the group consisting of hydrogen, nitro, amino, isothiocyanato, semicarbazido, thiosemicarbazido, carboxyl, bromoacetamido and maleimido;
R³ is selected from the group consisting of C₁-C₄ alkoxy, -OCH₂COOH, hydroxy and hydrogen;
R⁴ is selected from the group consisting of hydrogen, nitro, amino, isothiocyanato, semicarbazido, thiosemicarbazido, carboxyl, bromoacetamido and maleimido;
each R⁵ independently is hydrogen or C₁-C₄ alkyl;
with the proviso that R² and R⁴ cannot both be hydrogen but one of R² and R⁴ must be hydrogen; or
a pharmaceutically acceptable salt thereof.

10. The ²²⁵Ac conjugate of claim 6 wherein the functionalized chelant compound is 1-[(2-methoxy-5-isothiocyanatophenyl)-carboxymethyl]-4,7,10-triscarboxymethyl-1,4,7,10-tetraazacyclododecane (MeO-DOTA-NCS).

11. The ²²⁵Ac conjugate of any one of claims 6 to 10 wherein the biological molecule is an antibody or antibody fragment.

12. The ²²⁵Ac conjugate of any one of claims 6 to 10 wherein the biological molecule is selected from the group of antibodies consisting of NuM195, CC-49, CC-49 F(ab′)₂, CC-83, and CC-83 F(ab′)₂.

13. The ²²⁵Ac conjugate of claim 6 wherein the functionalized chelant compound of the conjugate is 1-[(2-methoxy-5-isothiocyanatophenyl)-carboxymethyl]-4,7,10-triscarboxy-methyl-1,4,7,10-tetraazacyclododecane and the biological molecule is selected from the group of antibodies consisting of HuM195, CC-49, CC-49 F(ab′)₂, CC-83, and CC-83 F(ab′)₂.

14. The ²²⁵Ac conjugate of claim 13 wherein the functionalized chelant compound of the conjugate is 1-[(2-methoxy-5-isothiocyanatophenyl)-carboxymethyl]-4,7,10-triscarboxy-methyl-1,4,7,10-tetraazacyclododecane and the biological molecule is HuM195 antibody.

15. A pharmaceutical formulation comprising the ²²⁵Ac conjugate of any one of claims 6 to 10 with a pharmaceutically acceptale carrier.

16. A pharmaceutical formulation comprising the ²²⁵Ac conjugate of claim 12 with a pharmaceutically acceptable carrier.

17. A pharmaceutical formulation comprising the ²²⁵Ac conjugate of claim 13 or claim 14 with a pharmaceutically acceptable carrier.

18. A method of the therapeutic treatment of a mammal having cancer which comprises administering to said mammal a therapeutically effective amount of a pharmaceutical formulation of claim 15.

19. A method of the therapeutic treatment of a mammal having cancer which comprises administering to said mammal a therapeutically effective amount of a pharmaceutical formulation of claim 16.

20. A method of the therapeutic treatment of a mammal having cancer which comprises administering to said mammal a therapeutically effective amount of a pharmaceutical formulation of claim 17.
